(19) United States
(12) Patent Application Publication
(10) Pub. No.: US 2024/0000897 A1
(43) Pub. Date: Jan. 4, 2024

(54) INJECTABLE BIODEGRADABLE POLYMERIC COMPLEX FOR GLUCOSE-RESPONSIVE INSULIN DELIVERY

(71) Applicant: THE REGENTS OF THE UNIVERSITY OF CALIFORNIA, Oakland, CA (US)

(72) Inventors: Zhen Gu, Los Angeles, CA (US); Jinqiang Wang, Los Angeles, CA (US)

(73) Assignee: THE REGENTS OF THE UNIVERSITY OF CALIFORNIA, Oakland, CA (US)

(21) Appl. No.: 18/254,166

(22) PCT Filed: Nov. 30, 2021
(86) PCT No.: PCT/US2021/061294

§ 371 Date: May 24, 2023

Related U.S. Application Data
(60) Provisional application No. 63/120,688, filed on Dec. 2, 2020.

(51) Int. Cl. A61K 38/28; A61K 47/34; A61P 3/10

(52) U.S. Cl. CPC A61K 38/28; A61K 47/34; A61P 3/10; A61K 9/0019

(57) ABSTRACT

A glucose-responsive therapeutic material demonstrates consistent and slow basal insulin release under a normoglycemic condition and accelerated insulin release in response to hyperglycemia. The therapeutic material uses a poly-L-lysine-derived polymer (PLL) modified with 4-carboxy-3-fluorophenylboronic acid (FPBA) that forms a polymer-insulin complex for glucose-stimulated insulin delivery. The release profile of the therapeutic material may be adjusted or tuned by altering the ratio of modified polymer (PLL-FPBA) to insulin in the therapeutic material, FPBA-modification degree of polymer, and altering the molecular weight of the polymer. The therapeutic material may be delivered to a mammalian subject using a delivery device (e.g., subcutaneous injection).

### RELATED APPLICATION

This Application claims priority to U.S. Provisional Patent Application No. 63/120,688 filed on Dec. 2, 2020, which is hereby incorporated by reference. Priority is claimed pursuant to 35 U.S.C. § 119 and any other applicable statute.

### TECHNICAL FIELD

The technical field relates an injectable and biodegradable glucose-responsive cationic polymer that forms polymer-insulin complexes for glucose-responsive insulin delivery. The polymer-insulin complexes may be injected, for example, subcutaneously into a mammalian subject for blood glucose regulation.

### BACKGROUND

Diabetes mellitus currently affects more than 463 million people worldwide and it is estimated to affect more than 700 million in 2045. Insulin replacement remains essential in treating type 1 and advanced type 2 diabetes. In healthy individuals, endogenous insulin secretion by β-cells of the pancreas oscillates synchronously with the fluctuation of blood glucose levels (BGLs), thereby minimizing both hyper- and hypoglycemia. Although exogenous insulin replacement strategies are designed to mimic endogenous insulin secretion, the daily administration of injected or infused insulin must be carefully titrated according to an individual's physiology and lifestyle, including changes in stress, physical activity, and dietary intake that may occur day by day. Moreover, excess doses of exogenous insulin can cause life-threatening hypoglycemia, thereby limiting its effectiveness in broad patient populations. Therefore, a synthetic system that can mimic β-cells by releasing insulin in a glucose-dependent manner is attractive for facilitating insulin administration by maximizing effectiveness and increasing safety. To date, various glucose-responsive insulin delivery systems, such as microneedles, hydrogels, nanoparticles or microparticles, complexes, liposomes, cells, and insulin analogs, have been extensively investigated. Among these systems, a glucose-responsive, charge-switchable complex has been validated with robust glucose-responsive performance in animal models. However, the non-biodegradable polymer backbone may bring long-term biocompatibility issues. Also, the normoglycemia state of diabetic mice treated with this formulation only maintained for up to eight (8) hours because of the fast basal insulin release rate, partially arising from the weak interaction between insulin and polymer due to the low molecular weight of the polymer. Therefore, the employment of a biodegradable cationic macromolecule with high molecular weight could potentially solve the biocompatibility issue and enhance the stability of insulin complex to reduce the basal insulin release rate. In addition, a high glucose stimulation index is also required to mimic the β-cell function for enhancing the blood glucose regulation ability. Because the complicated biological environment could alter the insulin release behavior from the complex, understanding the thermodynamics and kinetics of the in vitro glucose-responsive insulin release from the complex and the effect of the physical properties of the insulin complex, such as the arylboronic acid-modification degree and polymer-to-insulin ratio, on the relevant in vivo glucose stimulation index is essential in guiding the design and preparation of a clinically-translatable glucose-responsive insulin formulation. Eventually, this investigation could help build a bridge between in vitro insulin release rate and glucose-responsiveness and the in vivo blood glucose regulation ability and blood stimulated insulin release, respectively.

### SUMMARY

In one embodiment, an injectable and biodegradable glucose-responsive cationic polymer is disclosed that forms a polymer-insulin complex for glucose-responsive insulin delivery. The polymer-insulin complexes may be injected, for example, subcutaneously into a subject for blood glucose regulation. The cationic polymer is prepared by modifying fast-basal biodegradable poly-L-lysine (PLL) with 4-carboxy-3-fluorophenylboronic acid (FPBA), which is a widely used glucose-sensing component. Subsequently, these polymers are applied to prepare complexes with negatively charged insulin, whose isoelectronic point is pH 5.3 to 5.35, by leveraging electrostatic attraction at physiological pH. Since the driving force for the formation of polyion complex is also associated with the increase of entropy due to release of counterions, the stability of complex formed from positively-charged polymer chain and negatively-charged insulin could be affected by molecular weight (MW) of PLL, the FPBA modification degree, the polymer-to-insulin ratio, and the glucose concentration. In the presence of glucose, the binding of FPBA to glucose induces a decrease of the apparent pKₐ of FPBA moiety. Thus, introducing negative charges into the polymer chain and subsequentially reducing the positive charge density in polymer chains result in a decreased attraction between polymer and insulin mainly because of a reduced increase of entropy during the formation of complexes, consequently leading to a weakened binding between polymer and insulin and triggering insulin release from complexes (FIGS. 1C-1D). When subcutaneously injected in chemically-induced type 1 diabetic mice, the complexes deposit under the skin and release insulin slowly under a normoglycemic condition, maintaining euglycemia. Of course, the polymer-insulin complex may be injected into other mammals such as humans as a therapeutic. Upon intraperitoneal glucose injection to the complex-treated diabetic mice, elevated BGLs trigger insulin release from the subcutaneous complex, resulting in increased plasma insulin levels and correction of hyperglycemia. The impacts of the PBA-modification degree in the polymer and polymer-to-insulin ratio on the duration of normoglycemia and in vivo glucose-responsive performance may be tuned or adjusted depending on the patient and/or application.

In one embodiment, an injectable and biodegradable glucose-responsive material is disclosed that includes a poly-L-lysine (PLL) polymer modified with 4-carboxy-3-fluorophenylboronic acid (FPBA) that is loaded with insulin to form polymer-insulin complexes. In one embodiment, the modified polymer PLL-FPBA is loaded with insulin with the range of about 0.5 to about 1 times (on a weight basis) of the PLL-FPBA. In another embodiment, the modified polymer has the formula PLLₓ-FPBA_{y}, wherein x is in the range of about 0.2 to about 0.9 and y is in the range of about 0.8 to about 0.1.

In another embodiment, a kit may be provided that includes an injection or delivery device and the injectable and biodegradable glucose-responsive material.

In another embodiment, a method of using the injectable and biodegradable glucose-responsive material includes delivering a volume of the material to a subject. This may be done, for example, by injection (e.g., subcutaneous or intramuscular injection).

### BRIEF DESCRIPTION OF THE DRAWINGS

FIG. TA illustrates one example of a kit that includes the injectable and biodegradable glucose-responsive therapeutic material and delivery device (e.g., syringe).

FIG. 1B illustrates the subcutaneous delivery of the therapeutic material to a subject.

FIGS. 1C and 1D illustrate a schematic of the formation of the complex and mechanism of glucose-responsive insulin release. The positively charged polymer with glucose-sensing element forms complex with the negatively charged insulin. The binding between glucose and FPBA decreases the pKₐ of FPBA, introduces a negative charge, weakens the attraction between polymer and insulin, and consequently stimulates the insulin release and shifts the equilibrium to free insulin. The structure of the polymer is shown in FIG. 1D.

FIG. 1E shows representative images of the RhB-insulin and Cy5-labeled PLL_{0.4}-FPBA_{0.6} before and after forming complexes. The complex was prepared from either Rhodamine B-labeled insulin and unlabeled polymer or cyanine 5 (Cy5)-labeled PLL_{0.4}-FPBA_{0.6} and unlabeled insulin. Insulin and polymer were used in equal weight.

FIG. 1F shows representative fluorescence images of the complex. Cy5-labeled PLL_{0.4}-FPBA_{0.6} and RhB-insulin, respectively (and merged—right). Scale bar, 100 μm.

FIGS. 2A-2I illustrate the in vitro glucose-responsive insulin release from the glucose-responsive material. FIGS. 2A-2C include a schematic illustration of glucose binding and graphs showing change in glucose concentration over time. The glucose binds to the FPBA residues in polymers and leads to decreased glucose concentration in solution. The complexes were prepared from an equal weight of insulin and PLL_{0.65}-FPBA_{0.35} (LL1-insulin, FIG. 2B) and PLL_{0.4}-FPBA_{0.6} (L1-insulin, FIG. 2C), respectively. PLL used here had a MW of 30-70 kg/mol. The glucose concentration was measured using a glucose meter (Clarity).

FIGS. 2D-2F include a schematic illustration of insulin release from the complexes and graphs showing insulin release over time. The complex was prepared from an equal weight of insulin and either PLL_{0.65}-FPBA_{0.35} (LL1-insulin, FIG. 2E) or PLL_{0.4}-FPBA_{0.6} (L1-insulin, FIG. 2F). LL1 and L1 have N/C ratios of 3.5 and 2.1, respectively (see Table 1). The glucose-binding to FPBA weakened the attraction and liberated insulin from the complex into the solution. FIGS. 2G-2I include a schematic illustration of insulin release from the complexes and graphs showing insulin release over time. The complexes were prepared from insulin and either PLL_{0.65}-FPBA_{0.35} (LL2-insulin, FIG. 2H) or PLL_{0.4}-FPBA_{0.6} (L2-insulin, FIG. 2I) of double weight. LL2 and L2 have N/C ratios of 6.3 and 3.6, respectively (see Table 1). Data are mean±SD (n=3).

FIGS. 3A-3D illustrate in vivo studies in type 1 diabetic mice. FIG. 3A shows representative IVIS images of mice after treated with insulin and various insulin complexes. Insulin was labeled with Cy5. FIG. 3B illustrates the quantification of fluorescence intensity in (FIG. 3A). Data are mean±SD (n=3). SD, standard deviation. FIG. 3C illustrates blood glucose levels of diabetic mice treated with PBS, native insulin and insulin complexes that were prepared from an equal weight of native insulin and PLL_{0.57}-FPBA_{0.43} with the original PLL MW of 4-15 kg/mol or PLL_{0.6}-FPBA_{0.4} with original PLL MW of 15-30 kg/mol. Data are mean±SD (n=5 to 10). FIG. 3D shows blood glucose levels of diabetic mice treated with LL1-insulin, LL2-insulin, L1-insulin, and L2-insulin, with PLL having an original MW of 30-70 kg/mol. The insulin-equivalent dose was set to 1.5 mg/kg. Data are mean±SD (n=5 to 10).

FIGS. 4A-4D show plasma insulin level change associated with intraperitoneal glucose tolerance test in diabetic mice. The diabetic mice were treated with LL1-insulin (FIG. 4A), LL2-insulin (FIG. 4B), L1-insulin (FIG. 4C), and L2-insulin (FIG. 4D), respectively. The insulin-equivalent dose was set to 1.5 mg/kg. The glucose (3 g/kg) was given at 8 hours posttreatment with complexes. The plasma insulin level of each mouse just before treatment was set as 100%. The 0 min time point was set at the time of glucose injection. Data are mean SEM (n=5). One-way ANOVA with Tukey post-hoc tests was used to carry out multiple comparisons. *P<0.05; **P<0.01.

FIGS. 5A-5B illustrate representative images of H&E or Masson's trichrome staining sections. Diabetic mice were injected with various complexes and the skins at the treatment sites were obtained between time intervals. H&E staining (FIG. 5A) and Masson's trichrome staining (FIG. 5B) were performed. The images were taken on a microscope (Nikon, Ti-U). The skins without treatment were used as control samples. Black arrows indicate the injected complexes. Scale bars, 250 μm.

FIG. 6 schematically illustrates the injectable and biodegradable glucose-responsive material comprising a poly-L-lysine (PLL) polymer modified with 4-carboxy-3-fluorophenylboronic acid (FPBA) forming complexes with insulin. Under hyperglycemia conditions glucose causes the release of insulin from the material.

FIG. 7A illustrates the synthesis route of PLL-FPBA.

FIG. 7B illustrates the ¹H-NMR spectrum of FPBA modified PLL₄₋₁₅ₖ in D₂O with TFA to adjust its pH. About 43% of the amino groups in this polymer was reacted with FPBA-NHS.

FIG. 8 illustrates the ¹H-NMR spectrum of FPBA modified PLL₁₅₋₃₀ₖ in D₂O with TFA to adjust its pH. About 40% of the amino groups in this polymer was reacted with FPBA-NHS.

FIG. 9 illustrates the ¹H-NMR spectrum of FPBA modified PLL₃₀ₖ₋₇₀ₖ in D₂O with TFA to adjust its pH. About 35% of the amino groups in this polymer was reacted with FPBA-NHS.

FIG. 10 illustrates the ¹H-NMR spectrum of FPBA modified PLL₃₀₋₇₀ₖ in D₂O with TFA to adjust its pH. About 60% of the amino groups in this polymer was reacted with FPBA-NHS.

FIG. 11 illustrates the MALDI-TOF mass spectra of PLL_{0.4}-FPBA_{0.6} before (left) and after (right) enzyme digestion. The concentration of the polymer was 20 mg/mL, while the 0.1 mg/mL of trypsin was used. The polymer was incubated with trypsin at 37° C. overnight on a shaker (300 rpm).

FIG. 12 illustrates the encapsulation efficiency of insulin for various complexes. Data are mean±SD (n=3).

FIGS. 13A-13C illustrate fluorescence images of insulin complex. FIG. 13A is a representative image of complex from the channel of Cy5. FIG. 13B is a representative image of complex obtained from the channel of Rhodamine B. FIG. 13C is a merge of the images of FIGS. 13A and 13B.

FIGS. 14A-14E illustrate representative SEM and TEM images of L2-insulin. (FIGS. 14A-14B) Representative SEM images of L2-insulin. Scale bars are 10 μm (FIG. 14A) and 5 μm (FIG. 14B), respectively. (FIGS. 14C-14E) Representative TEM images of L2-insulin. The complex was stained by phosphotungstic acid (2%). Complex particles with varied sizes were shown here. Scale bars are 1 μm (FIG. 14C), 0.5 μm (FIG. 14D), and 0.2 μm (FIG. 14E), respectively.

FIG. 15 illustrates glucose meter reading as a function of glucose concentration. Data are mean±SD (n=3).

FIG. 16 illustrates glucose-responsive insulin release from complex prepared from an equal weight of insulin and PLL_{0.57}-FPBA_{0.43} with the original PLL MW of 4-15 kg/mol. Data are mean±SD (n=3).

FIG. 17 illustrates glucose-responsive insulin release from complex prepared from an equal weight of insulin and PLL_{0.6}-FPBA_{0.4} with the original PLL MW of 15-30 kg/mol. Data are mean±SD (n=3).

FIG. 18 illustrates the glucose-dependent solubility of PLL_{0.4}-FPBA_{0.6} in PBS at pH 7.4. The original PLL has a MW of 30-70 kg/mol. The supernatant was centrifuged, collected, and measured using a Coomassie protein assay reagent. The apparent insulin level was calculated according to the standard curve of insulin. Data are mean±SD (n=3).

FIG. 19 illustrates the glucose-dependent solubility of PLL_{0.65}-FPBA_{0.35} in PBS at pH 7.4. The original PLL has a MW of 30-70 kg/mol. The supernatant was centrifuged, collected, and measured using a Coomassie protein assay reagent. The apparent insulin level was calculated according to the standard curve of insulin. Data are mean±SD (n=3).

FIG. 20 illustrates the glucose-dependent insulin release from bulk L1-insulin. L1-insulin was centrifuged (21, 000 G, 10 min) to the bottom of Eppendorf tubes. The complex stayed as a bulk at the bottom during the whole experiment. Data are mean+SD (n=3). Legend concentrations (0 mg/dL, 100 mg/dL, 200 mg/dL, 400 mg/dL) are represented left-to-right in histogram.

FIG. 21 illustrates the glucose-dependent insulin release from bulk L2-insulin. L1-insulin was centrifuged (21, 000 G, 10 min) to the bottom of Eppendorf tubes. The complex stayed as a bulk at the bottom during the whole experiment. Data are mean+SD (n=3). Legend concentrations (0 mg/dL, 100 mg/dL, 200 mg/dL, 400 mg/dL) are represented left-to-right in histogram.

FIG. 22 illustrates the dose-dependent blood glucose regulation ability of L1-insulin. Data are mean+SD (n=5). 0.5 mg/kg is top graph; 1.0 mg/kg is middle graph; 1.5 mg/kg is bottom graph.

FIG. 23 illustrates the statistical analysis of fluorescence intensity. Data are mean±SD (n=3). Two-way ANOVA was used to calculate the difference among difference groups. Only P values with significant difference were shown. *P<0.05, **P<0.01, ***P<0.001, ****P<0.0001. Legend (Insulin, LL1-insulin, LL2-insulin, L1-insulin, L2-insulin) are represented left-to-right in histogram.

FIGS. 24A-24C illustrate intraperitoneal glucose tolerance tests. Healthy mice (FIG. 24A) and diabetic mice receiving native insulin (FIG. 24B) or PBS (FIG. 24C) were used as control groups. The insulin-equivalent dose was set to 1.5 mg/kg. The glucose (3 g/kg) was given at 8 hours posttreatment with complexes. Data are mean+SD (n=5).

FIG. 25 illustrates the dose-dependent cytotoxicity of PLL₃₀₋₇₀ₖ, PLL_{0.4}-FPBA_{0.6}, and PLL_{0.65}-FPBA_{0.35}. The cytotoxicity was evaluated on L929 murine fibroblast cells. Data are mean+SD (n=3). Legend (PLL, PLL_{0.4}-FPBA_{0.6}, PLL_{0.65}-FPBA_{0.35}) are represented left-to-right in histogram.

FIGS. 26A-26B illustrate the biodistribution of the polymer after subcutaneous injection. Polymers were labeled with Cy5 and formed complexes LL2-insulin and L2-insulin before injection (1.5 mg/kg insulin-eq. dose). The organs (FIG. 26A) and the skins (FIG. 26B) were obtained between time intervals. IVIS spectrum was used to measure the fluorescence of each organ. H, heart; Li, liver; S, spleen; Lu, lung; K, kidney. Dn, n^{th} day posttreatment; Wn, n^{th} week posttreatment; Con., control group without treatment.

FIG. 27 illustrates the biodistribution of the polymer after subcutaneous injection (PLL_{0.65}-FPBA_{0.35}, PLL_{0.4}-FPBA_{0.6}). Polymers were labeled with Cy5 and formed LL2-insulin and L2-insulin complexes before injection (1.5 mg/kg insulin eq. dose). The main organs were obtained between time intervals. IVIS spectrum was used to measure the fluorescence of each organ. H, heart; Li, liver; S, spleen; Lu, lung; K, kidney.

FIG. 28 illustrates the effect of complex treatment on blood cell count. Diabetic mice were treated with LL2-insulin and L2-insulin every two days at a dose of 1.5 mg/kg. Diabetic mice receiving PBS and healthy mice were used as control groups. Data are mean SD (n=5). RBC, red blood cell; PLT, platelet; WBC, white blood cell; NEUT, neutrophil; LYMPH, lymphocyte; MONO, monocyte; EO, eosinophil; BASO, basophil. Legend (Healthy, PBS, LL2-insulin, L2-insulin) are represented left-to-right in histogram.

FIG. 29 illustrates the effect of complex treatment on serum biochemical parameters indicating main organ healthiness. Diabetic mice were treated with LL2-insulin and L2-insulin every two days at a dose of 1.5 mg/kg for one week. Diabetic mice receiving PBS and healthy mice were used as control groups. Data are mean+SD (n=5). ALP, alkaline phosphatase; AST, aspartate transaminase; ALT, alanine transarninase: BUN, blood urea nitrogen. Legend (Healthy, Untreated, LL2-insulin, L2-insulin) are represented left-to-right in histogram.

### DETAILED DESCRIPTION OF ILLUSTRATED EMBODIMENTS

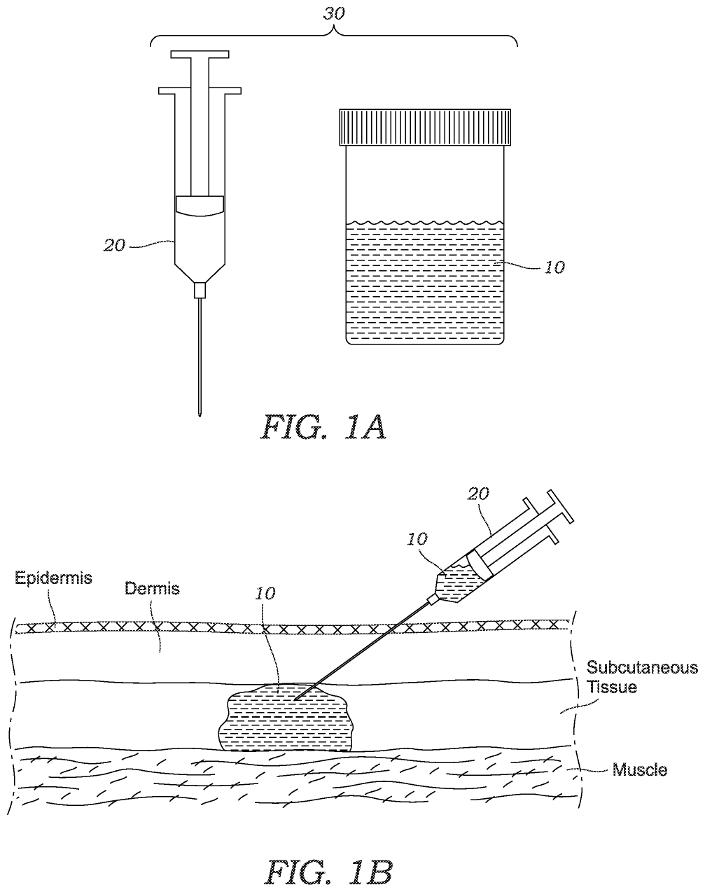

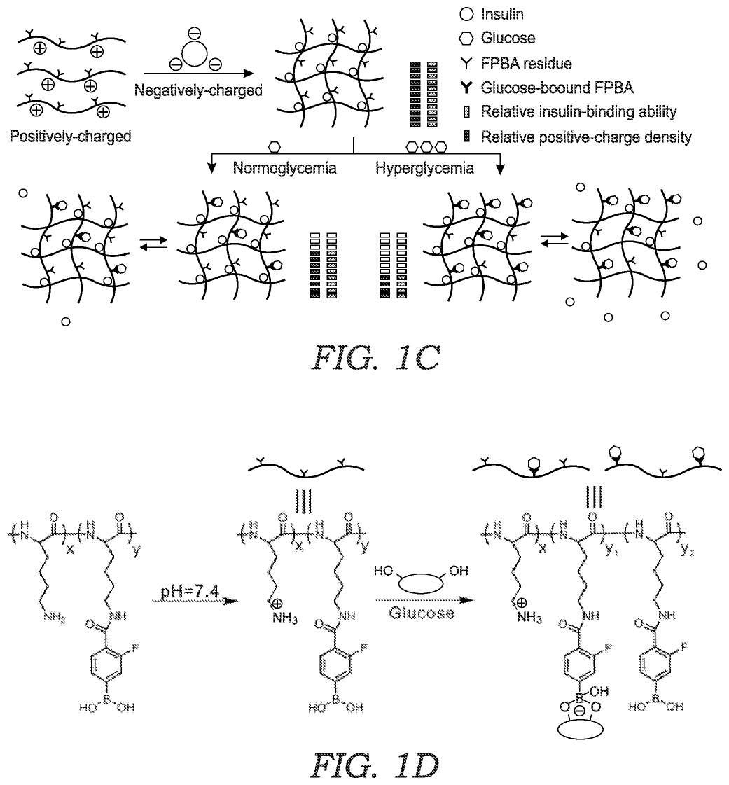

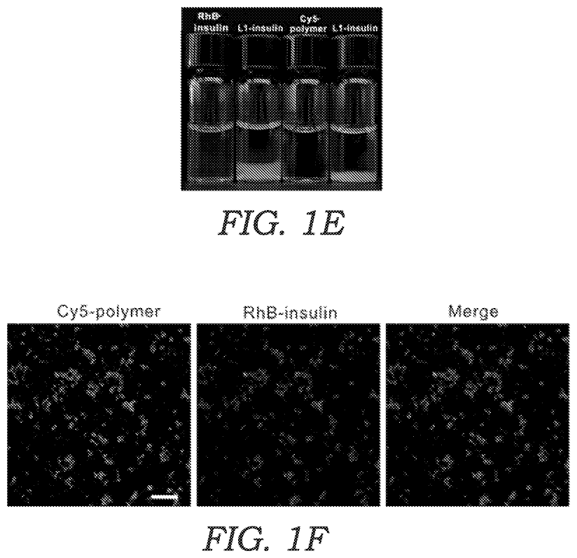

In one embodiment, and with reference to FIGS. 1A and TB, an injectable and biodegradable glucose-responsive material 10 is disclosed. The therapeutic material 10 is formed with a poly-L-lysine (PLL) polymer modified with 4-carboxy-3-fluorophenylboronic acid (FPBA) (a modified polymer PLL-FPBA) that forms a complex with insulin. The polymer-insulin complex that forms the therapeutic material 10 may then be administered to a subject (e.g., mammalian subject) to regulate glucose levels. The modified polymer PLL-FPBA material is loaded with insulin to form a complex. The amount or degree of loading of insulin may vary. For example, in one embodiment, the modified polymer PLL-FPBA is loaded with about an equal (weight) amount of insulin to generate the therapeutic material 10. In another embodiment, the amount of insulin (weight) is about half the amount of modified polymer PLL-FPBA (e.g., insulin: modified polymer PLL-FPBA is about 1:2). Of course, the amount of loading of insulin may also fall within this range (e.g., equal to half). Some other embodiments may have even less than equal or more than two times the amount of polymer to insulin. In one particular embodiment, the modified polymer has the chemical formula PLLₓ-FPBA_{y}, wherein x is in the range of about 0.2 to about 0.9 and y is in the range of about 0.8 to about 0.1. In another embodiment, x is in the range of about 0.4 to about 0.65 and y is in the range of about 0.6 to about 0.35.

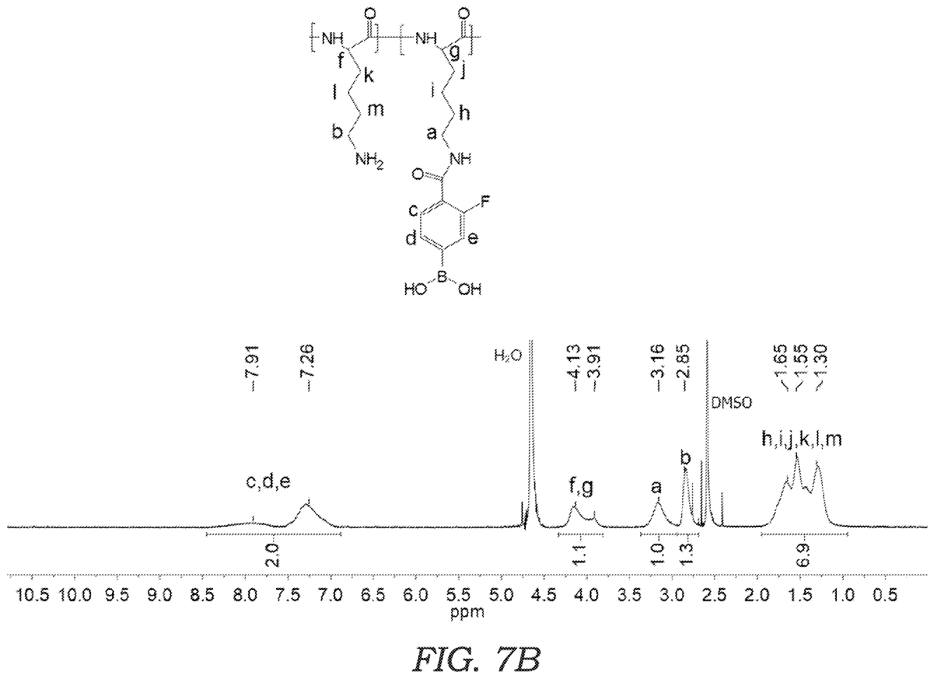

To make the therapeutic material 10, PLL is modified with FPBA as illustrated in FIG. 7A and described in Wang et al., Charge-Switchable Polymeric Complex for Glucose-Responsive Insulin Delivery in Mice and Pigs, Sci. Adv, 5(7), eaaw4357 (2019), which is incorporated herein by reference. Polymer-insulin complexes were then prepared by mixing insulin and PLL-FPBA in an acidic solution (pH=2), followed by an instant adjustment of pH to around 7.4. At this pH, insulin is negatively charged while PLL-FPBA is positively charged, facilitating the generation of a stable complex.

The therapeutic material 10 may be administered to a subject using a delivery device 20 as illustrated in FIGS. 1A and 1B. The delivery device 20 may include, for example, an injection device such as a syringe. For example, the therapeutic material 10 may be provided as part of a kit 30 with the delivery device 20 (e.g., syringe or other injection device) along with the therapeutic material 10. The therapeutic material 10 may be preloaded in the delivery device 20 or contained separately which is then loaded into the delivery device 20. The therapeutic material 10 may be suspended or contained in a buffer solution such as phosphate-buffered saline (PBS). The therapeutic material 10 may then be delivered to the subject by injection using the delivery device 20 as illustrated in FIG. 1B. A volume of the therapeutic material 10 may be injected into the subcutaneous tissue (i.e., subcutaneous injection or even the muscle tissue (i.e., intramuscular injection). The therapeutic material 10 may be injected at a single location or multiple locations. The therapeutic material 10 is biodegradable over time. Because of the biodegradable nature of the therapeutic material 10, in some embodiments, the subject may have to periodically visit their physician or other medical professional to administer additional injections. The therapeutic material 10 may be used to treat type 1 and/or type 2 diabetics. The therapeutic material has particular 10 applicability to treat hyperglycemia conditions.

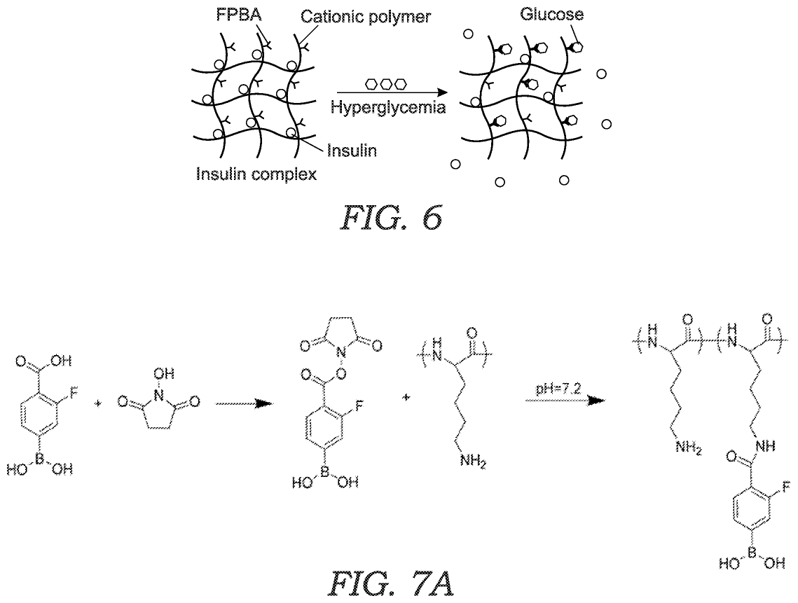

FIGS. 1C, 1D and 6 schematically illustrate the operation of the therapeutic material 10. As seen in FIG. 1C, the positively charged modified polymer PLL-FPBA forms a complex with the negatively charged insulin. In the presence of glucose, the binding of FPBA to glucose induces a decrease of the apparent pKₐ of the FPBA moiety of the modified polymer. Thus, introducing negative charges into the modified polymer PLL-FPBA chain and subsequentially reducing the positive charge density in the polymer chains result in a decreased attraction between the modified polymer PLL-FPBA and insulin, consequently leading to a weakened binding between the modified polymer and insulin and triggering insulin release from complexes. This is illustrated in FIG. 1C for the normoglycemia and hyperglycemia states (see also FIG. 6),

### EXPERIMENTAL

Results and Discussion

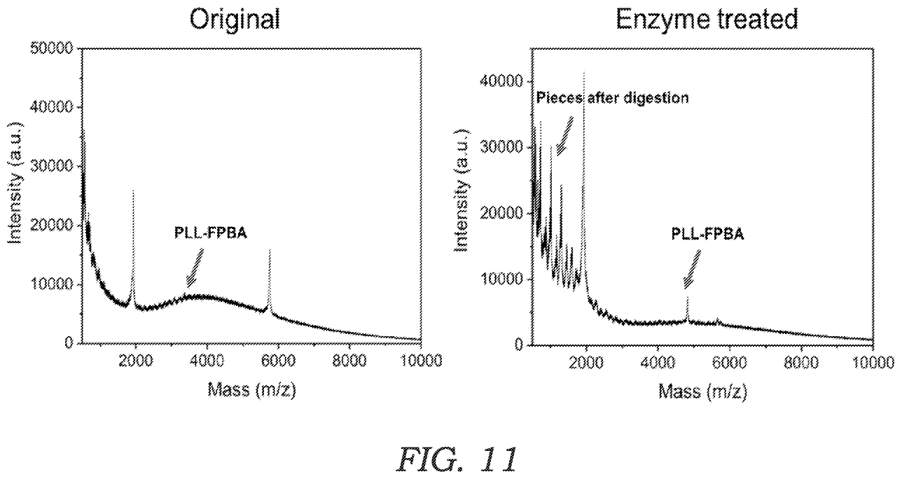

PLL is abundant in amino groups and was modified with FPBA using the methods previously described in Wang et al., as discussed herein and illustrated in FIG. 7A. The chemical structures of the obtained FPBA-modified PLL (PLL-FPBA) were characterized by ¹H-NMR (FIGS. 7B and 8-10). PLL-FPBA could be hydrolyzed to relatively small molecules when exposed to the widely-used model enzyme trypsin as confirmed by matrix-assisted laser desorption ionization-time of flight mass spectrometry (MALDI-TOF, FIG. 11). Insulin complexes were prepared by mixing insulin and PLL-FPBA in an acidic solution (pH=2), followed by an instant adjustment of pH to 7.4. At this physiological-relevant pH, insulin is negatively charged while PLL-FPBA is positively charged, facilitating the generation of a polyion complex. Herein, the experiments were mainly focused on four complexes prepared from PLL-FPBA with an original PLL MW of 30-70 kg/mol. For simplicity, the complexes prepared from insulin and equal or two-fold weight of PLL_{0.65}-FPBA_{0.35} (with 35% of amino groups reacted with FPBA-NHS) are designated as LL1-insulin and LL2-insulin (LLn-insulin: the first L indicated Lower-FPBA content; LLn-insulin, the second L indicated L-Lysine; LLn-insulin, the n indicated the weight ratio between polymer and insulin), while the complexes prepared from insulin and equal or two-fold weight of PLL_{0.4}-FPBA_{0.6} (with 60% of amino groups reacted with FPBA-NHS) are designated as L1-insulin and L2-insulin (Ln-insulin: the L indicated L-Lysine), respectively (Table 1).

TABLE 1

| | | Polymer | N/C ratio | | | | |
|---|---|---|---|---|---|---|---|
| | | to-insulin | (N, amine; | Z-averaged | Zeta-potential (mV) in | | |
| | | ratio | C, carboxylic | size | glucose solution (mg/dL) | | |
| Polymer | Abbreviation | (wt./wt.) | acid) | (nm) | 0 | 100 | 400 |
| PLL_{0.65}- | LL1-insulin | 1 | 3.5 | 4569 ± 632 | 16.2 ± 4.2 | 10.0 ± 4.1 | 6.9 ± 1.0 |
| FPBA_{0.35} | LL2-insulin | 2 | 6.3 | 2527 ± 1217 | 20.9 ± 9.1 | 11.0 ± 2.1 | 7.2 ± 6.5 |
| PLL_{0.4}- | L1-insulin | 1 | 2.1 | 3295 ± 1476 | −2.5 ± 5.0 | −7.0 ± 1.1 | −11.9 ± 2.2 |
| FPBA_{0.6} | L2-insulin | 2 | 3.6 | 2451 ± 1470 | −1.6 ± 4.7 | −10.0 ± 2.6 | −16.1 ± 1.2 |

Each insulin molecule has six carboxylic acid groups, three amino groups, and one guanidino group, which were all included in the calculation of N/C ratio. Phenylboronic acid groups were not included in the calculation even though they may carry negative charges. Data are presented in Table 1 as mean+SD (n=3).

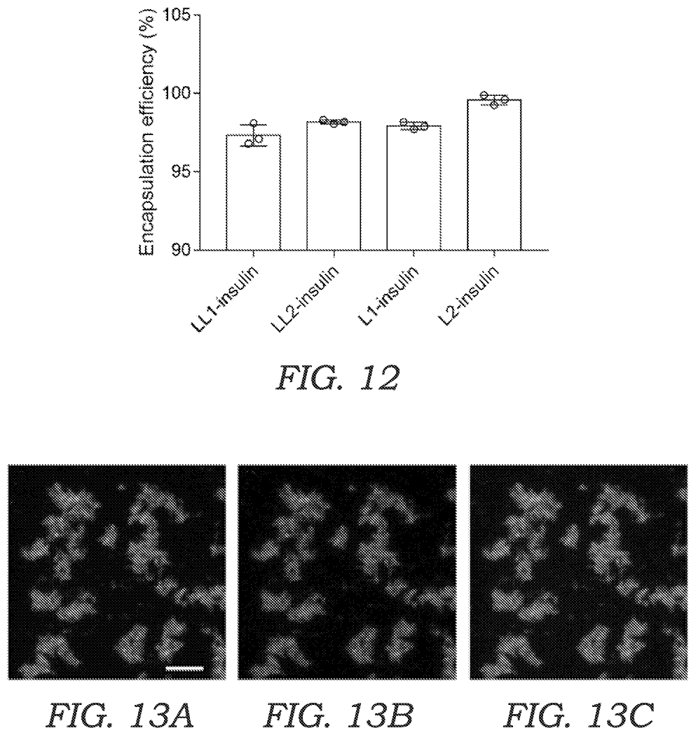

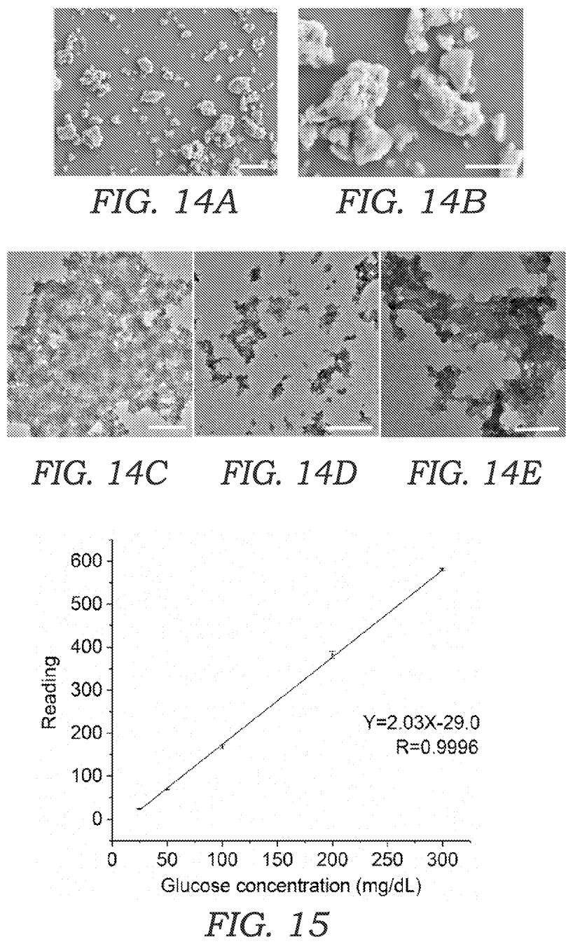

The loading efficiency of insulin was higher than 90% for these four complexes (FIG. 12). The micro-sized insulin complex displayed as a floc-like precipitate (FIG. 1E). Its morphology was further determined by fluorescence microscopy (FIG. 1F, and FIGS. 13A-13C), transmission electron microscopy (TEM), and scanning electron microscopy (SEM) (FIGS. 14A-14E). The hydrodynamic size and zeta-potential of these complex particles were also measured (Table 1 herein).

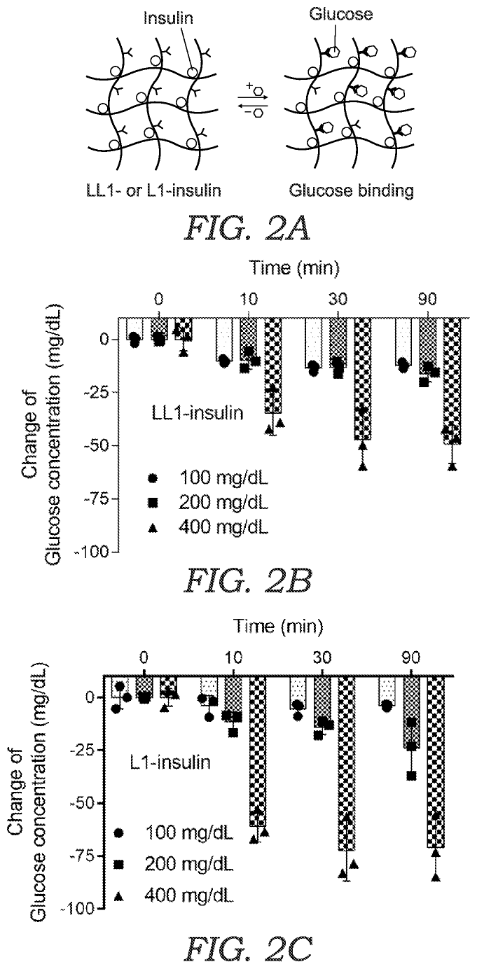

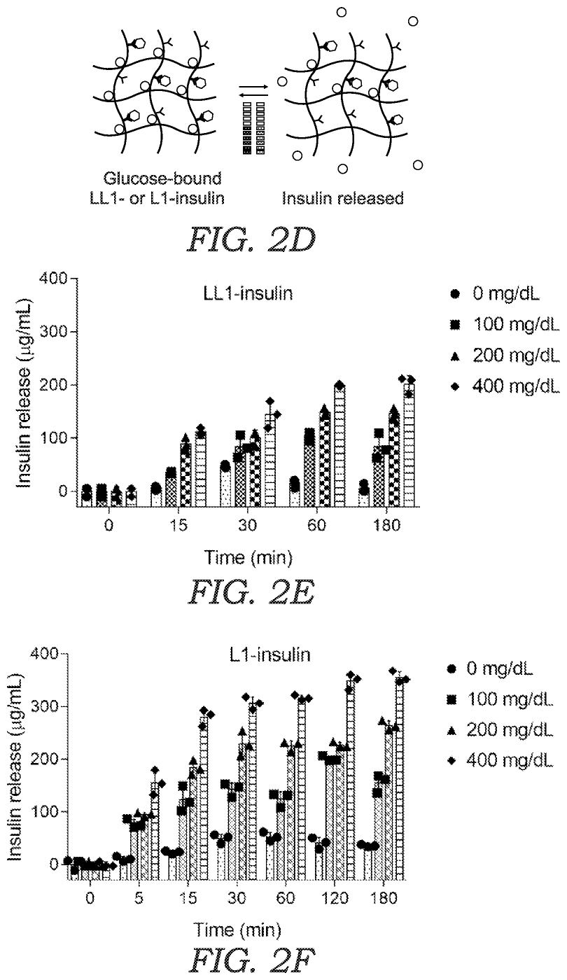

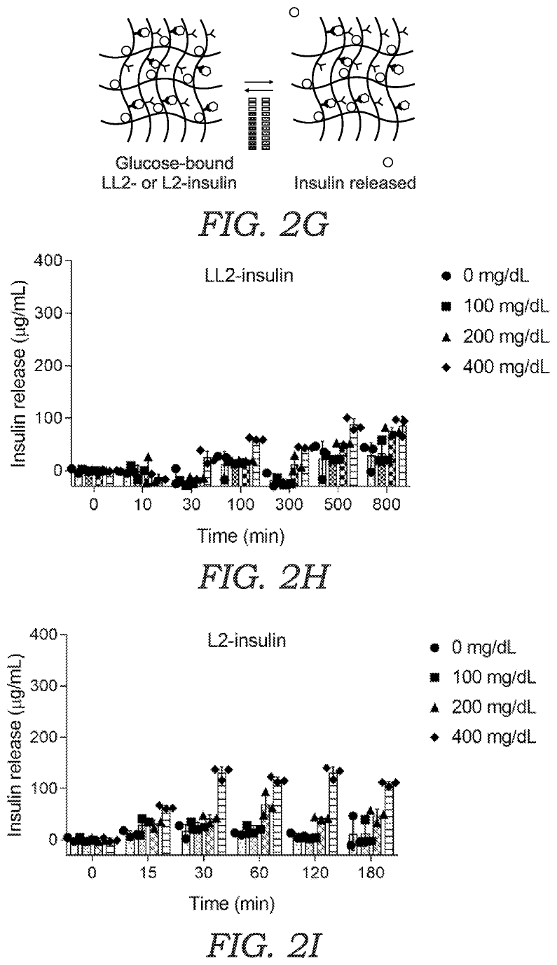

The glucose-binding ability of the FPBA element in modified polymers was evaluated in phosphate-buffered saline at pH 7.4 (PBS 7.4) with varying glucose concentrations (100, 200 and 400 mg/dL) (FIGS. 2A-2C). The glucose concentration was measured using a glucose meter by establishing a standard curve (FIG. 15). An instant decrease in glucose concentration was observed once the glucose was added to the complex suspension, suggesting fast glucose binding (FIGS. 2B-2C). Also, the glucose binding to FPBA increased over time for both complexes. LL1-insulin (FIG. 2B) showed similar glucose-binding capacity with L1-insulin (FIG. 2C) at glucose concentrations of 100 and 200 mg/dL, indicating that the high density of positive charge in PLL_{0.65}-FPBA_{0.35} may facilitate FPBA binding to glucose. Meanwhile, a lower glucose-binding capacity of LL1-insulin than that of L1-insulin was observed at 400 mg/dL glucose solution (FIGS. 2B-2C).

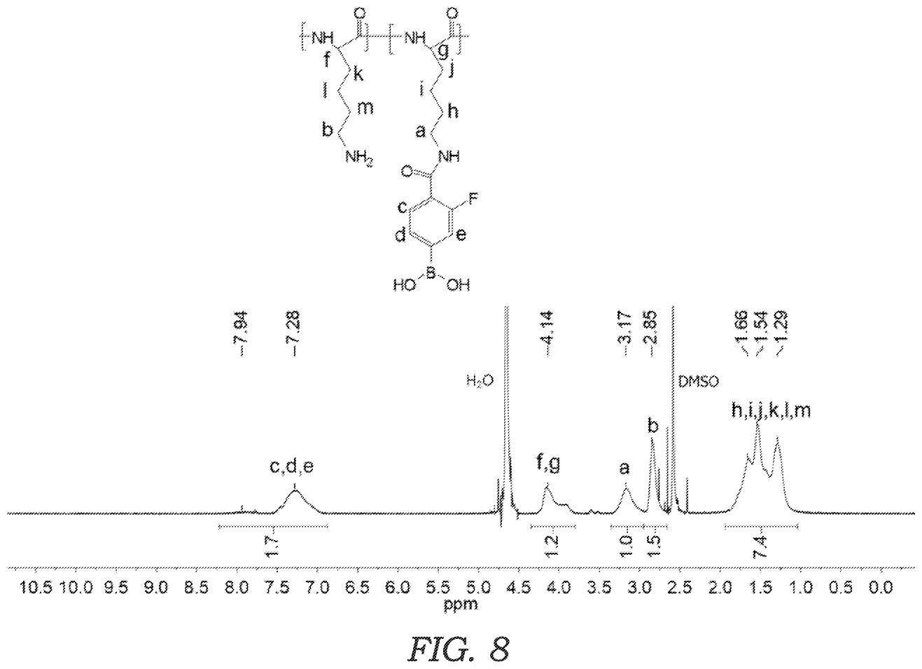

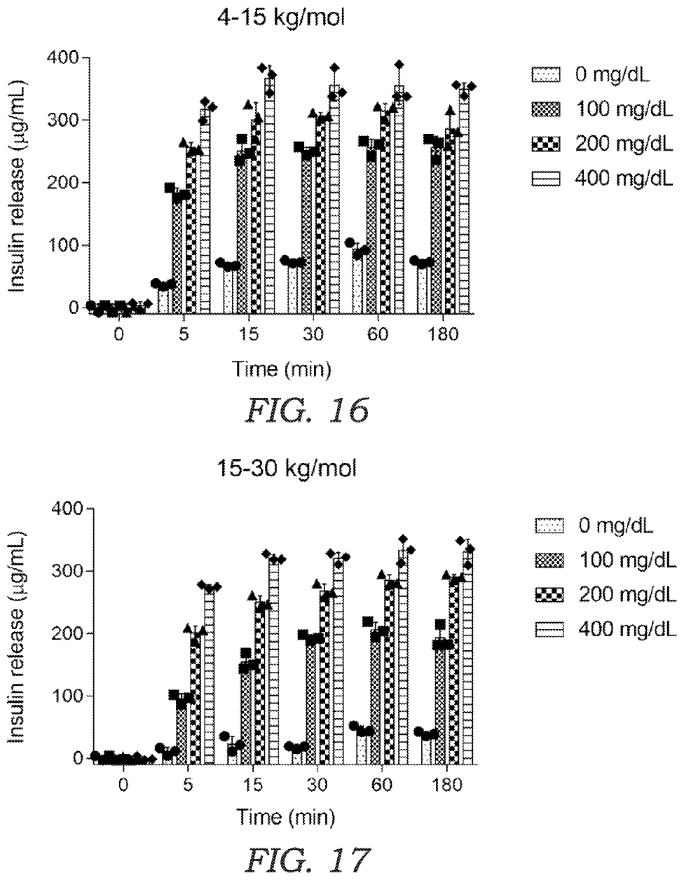

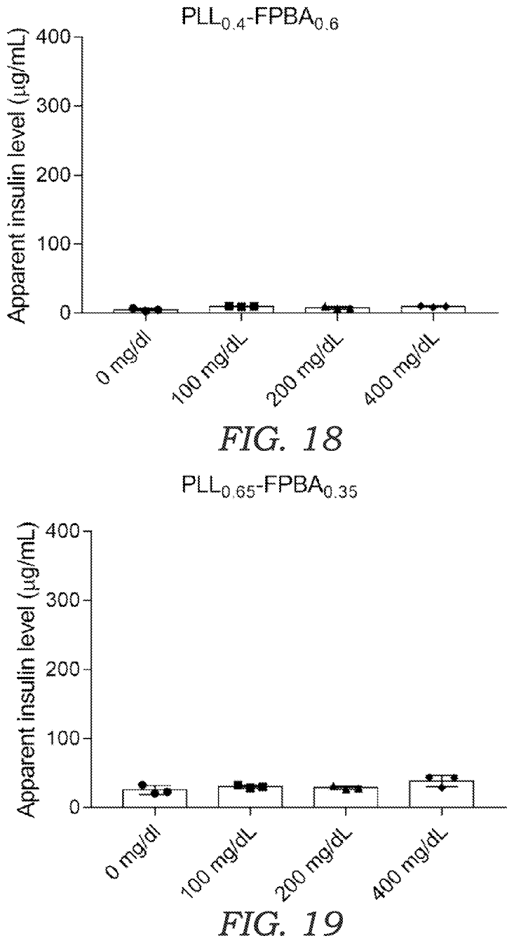

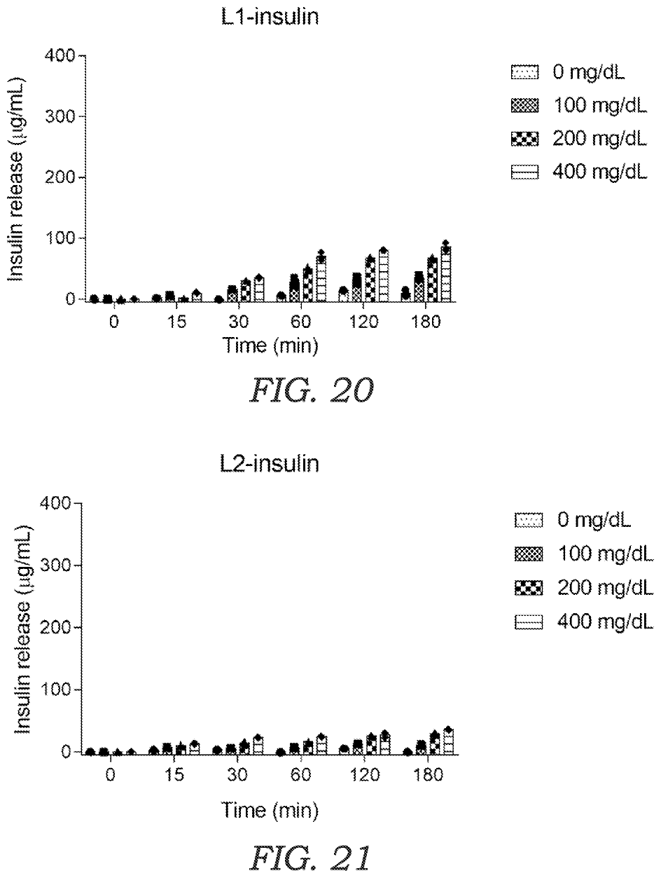

The glucose-responsive insulin release performance of these complexes was evaluated in PBS 7.4 with varying glucose concentrations (FIGS. 2D-2I and FIGS. 16 and 17). The insulin concentration was measured using Coomassie protein assay reagent. PLL-FPBA was poorly soluble in PBS 7.4, so it caused negligible interference (FIGS. 18-19). Insulin released at a slow rate in PBS 7.4 without glucose, and free insulin reached equilibrium at a concentration lower than 50 μg/mL for LL1-insulin, LL2-insulin, L1-insulin, and L2-insulin. Following the addition of glucose to PBS solution, the rate of insulin release and the equilibrated free insulin concentration increased. A higher glucose concentration could lead to more glucose binding to FPBA residuals on polymers, therefore leading to decreased positive charge density and reduced attraction between insulin and polymer. Of note, because both PLL-FPBA and complexes were precipitates, so the stability of the complex was monitored by measuring the insulin concentrations in supernatant of the complex suspension. The insulin release performance was further affected by the FPBA-modification degree, polymer-to-insulin ratio, and polymer MW as follows. First, increasing glucose levels promoted insulin release via introducing negative charges, leading to the reduced attraction between polymer chain and insulin. For example, the insulin release from LL1-insulin and L1-insulin reached 80 μg/mL and 140 μg/mL at 100 mg/dL glucose solution within 30 min, respectively, compared to 144 μg/mL and 305 μg/mL at 400 mg/dL glucose solution, respectively (FIGS. 2E-2F). Second, a higher FPBA content in the polymer means a lower positive charge density, so the binding of FPBA to glucose could induce a higher degree of switch of the positive charge. Also, a higher FPBA content indicated higher glucose binding capability especially at 400 mg/dL glucose solution (FIGS. 2B-2C), therefore introducing more negative charges and promoting greater insulin release. For example, the rate of insulin release from L1-insulin (FIG. 2F) exceeded that of LL1-insulin (FIG. 2E). A similar trend in insulin release rate was also observed when comparing LL2-insulin with L2-insulin, especially in 400 mg/dL glucose solution (FIGS. 2H-2I). Third, the ratio of polymer-to-insulin affected the insulin release rate. A higher polymer ratio could achieve a higher binding capacity of polymer to insulin, therefore reducing the basal free insulin level. After increasing the polymer-to-insulin ratio from one (for LL1-insulin and L1-insulin) to two (for LL2-insulin and L2-insulin), insulin molecules were held by two-fold positively-charged polymer chains, leading to enhanced binding between polymer chains and insulin molecules (FIG. 2D, 2G). This enhanced binding could slow down the release of insulin from complex and reduce free insulin level in solution. So, the free insulin quantity at equilibrium in LL2-insulin and L2-insulin suspensions was around 50% less than that of their associated counterparts with fewer polymer contents (FIGS. 2E, 2F, 2H, 2I). Among the four complexes prepared from PLL of 30-70 kg/mol, L2-insulin exhibited best glucose responsiveness regarding the ratio of free insulin concentrations of the complex suspension with 400 mg/dL glucose solution to that with 100 mg/dL glucose solution. The equilibrated free insulin concentration in the L2-insulin suspension at 400 mg/dL was 108 μg/mL, which is almost ten-fold to that at 100 mg/dL (FIG. 2I). In comparison, other complexes only achieved a ratio of around two. Finally, the molecular weight of polymer also had an impact on the insulin release rate and balanced insulin level. Compared with LL1-insulin with an original PLL of 30-70 kg/mol, insulin complexes prepared from PLL_{0.57}-FPBA_{0.43} (PLL of 4-15 kg/mol) and PLL_{0.6}-FPBA_{0.4} (PLL of 15-30 kg/mol—FIG. 8) had faster insulin release rates and higher balanced insulin levels at various glucose concentrations (FIGS. 16-17). Of note, the size of the complexes may also affect the insulin release, even though the size of the complex was not controlled. However, it was found that the insulin release from bulk L1-insulin and L2-insulin centrifuged to the bottom of the Eppendorf tube was slowed down as compared to their suspended counterparts (FIGS. 20-21). This reduced insulin release from complex with large size may result from the reduced diffusion rate of glucose into the complex and of insulin to the outside.

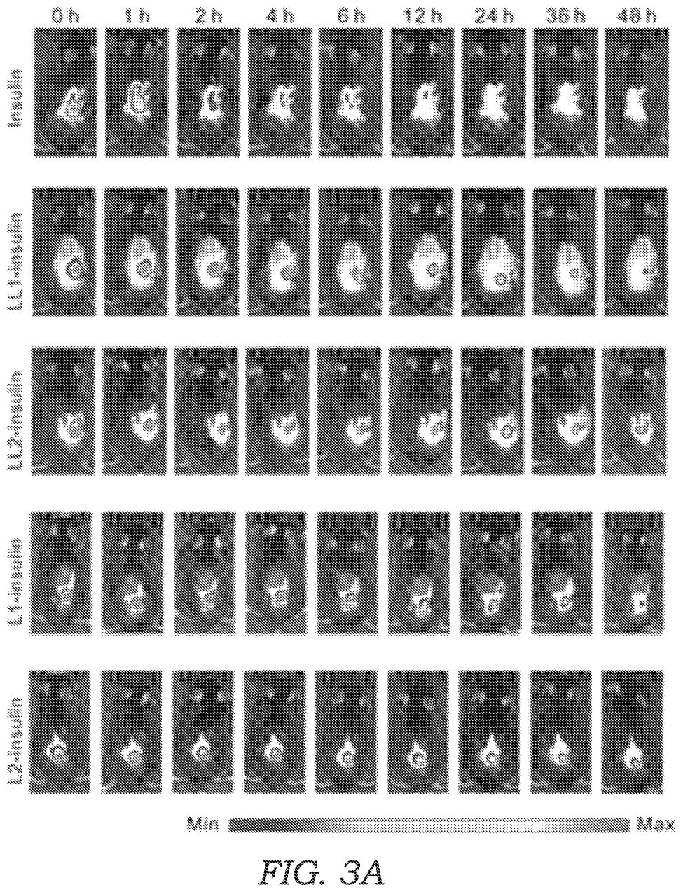

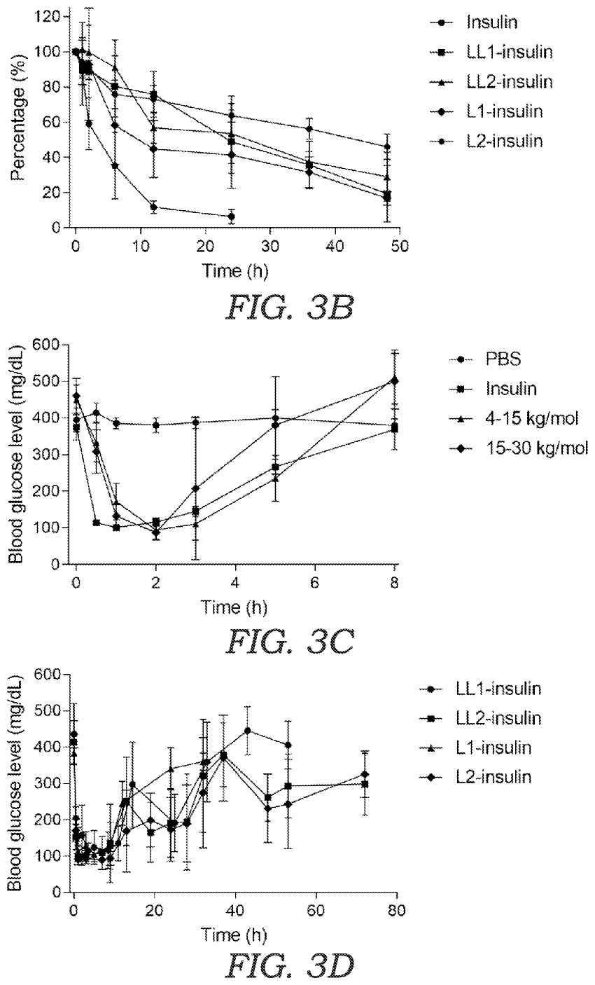

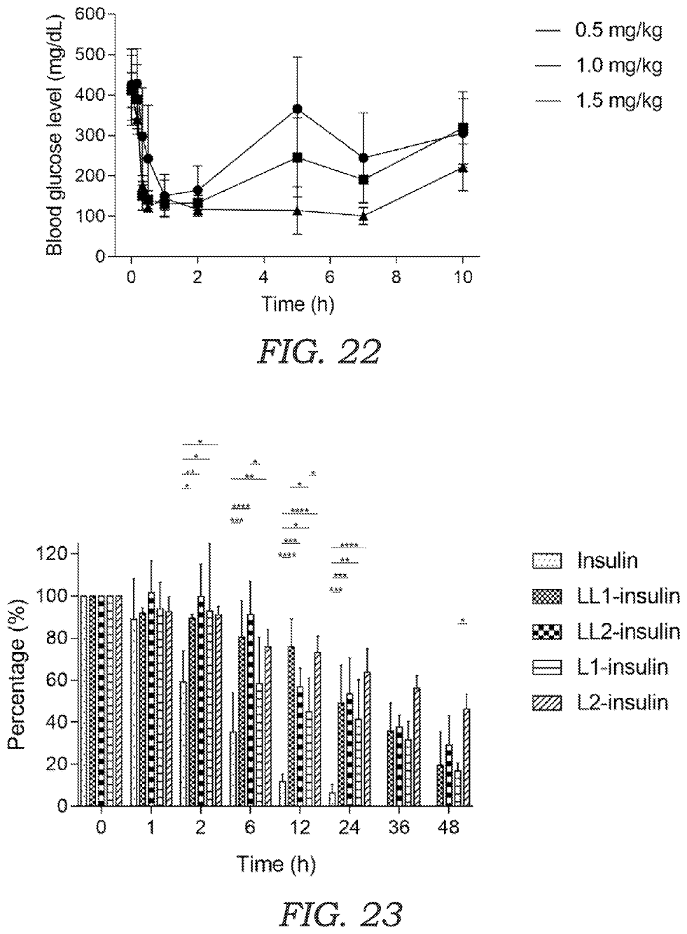

The in vivo blood glucose regulation ability of the insulin complexes was evaluated in C57BL/6J mice with type 1 diabetes induced by streptozotocin (STZ). Based on preliminary studies, the insulin equivalent dose was established as 1.5 mg/kg (FIG. 22). In each group, five to ten diabetic mice were included. Both the native insulin and complexes were subcutaneously injected. LL1-, LL2-, L1-, and L2-insulin all exhibited a longer retention time than free insulin based on in vivo imaging (FIGS. 3A-3B, FIG. 23). After subcutaneous injection, the BGLs of diabetic mice receiving injections of either complexes or insulin all decreased to normoglycemic levels (FIGS. 3C-3D). Theoretically, L1-insulin had a fastest in vitro insulin release rate while LL2-insulin had the slowest insulin release rate, so L1-insulin should achieve normoglycemia fastest while LL2-insulin should be the slowest one. However, the four formulations achieved normoglycemia in treated mice all at around 0.5 h may arise from the heterogeneity of insulin sensitivity among diabetic mice and the anesthesia procedure during insulin complex injection. Also, after injection, the complex could not sense the level of the interstitial glucose immediately because the complexes were suspended in PBS, the absorption of which took times and could delay the establishment of a local bio-relevant glucose environment surrounding the complex.

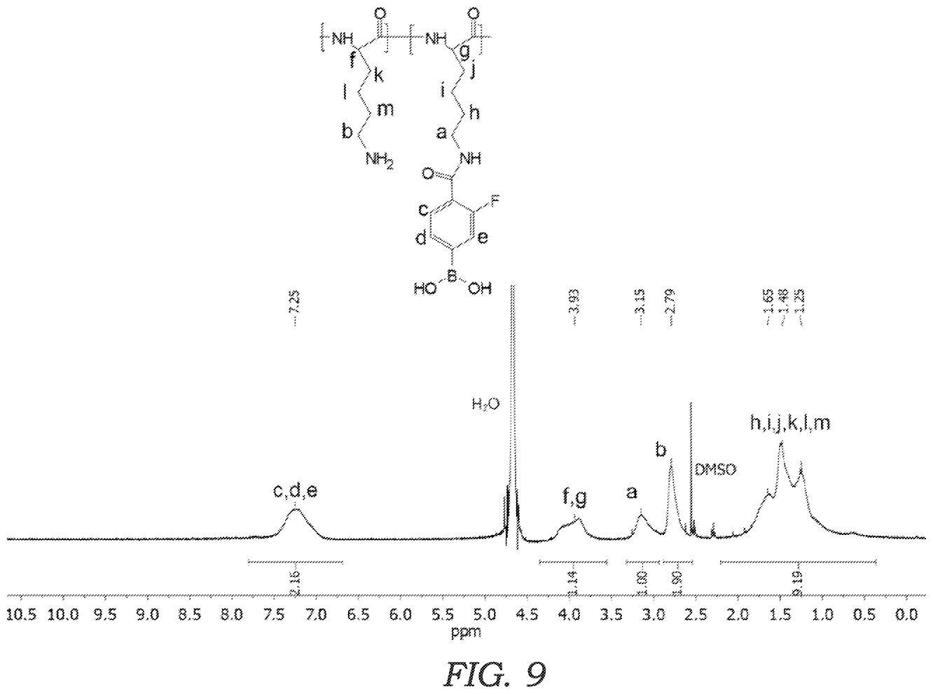

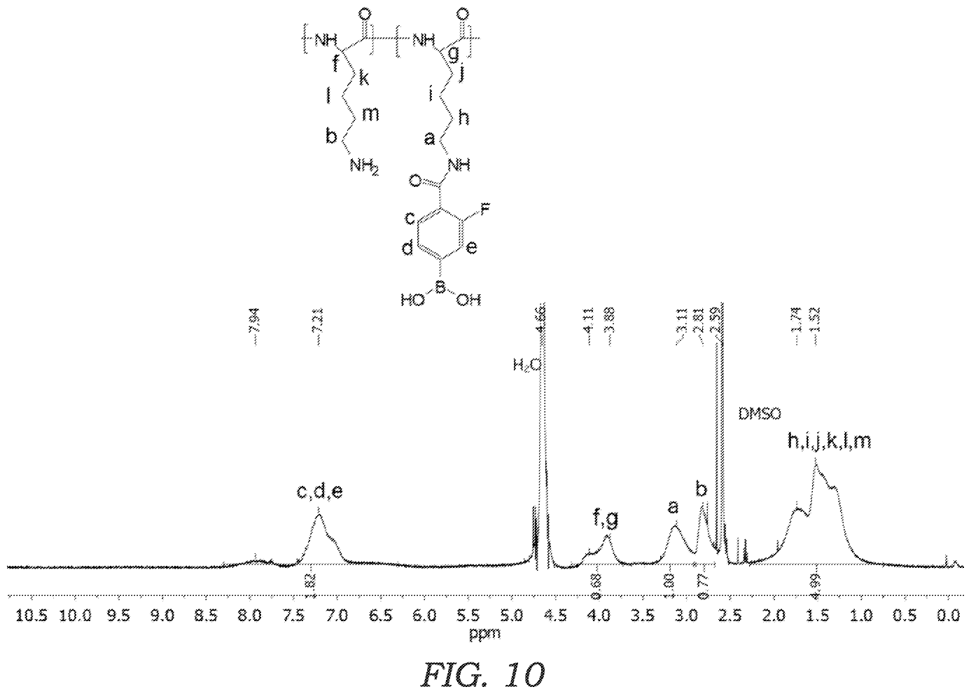

The normoglycemia duration of diabetic mice treated with complexes was affected by several factors. First, the MW of PLL greatly impacted the blood glucose regulation ability of the insulin complexes. For instance, the BGLs of the diabetic mice treated with complexes prepared from insulin and FPBA-modified PLL₄₋₁₅ₖ (43% FPBA modification) showed BGLs within the normal range for only five hours and returned to initial hyperglycemic levels 8 hours posttreatment (FIG. 3C). Increasing the MW of PLL to 15-30 kg/mol did not prolong the normoglycemia time (FIG. 3C). However, further increasing the MW of PLL to 30-70 kg/mol (FIGS. 9 and 10) achieved a normoglycemic state longer than 10 hours in diabetic mice treated with LL1-insulin, while BGLs returned to initial hyperglycemic levels after 43 hours post-treatment (FIG. 3D). Second, the FPBA-modification degree mattered, especially when there was an equal weight of insulin and polymer in the complex. Compared with LL1-insulin, L1-insulin only achieved normoglycemic BGLs for approximately 10 hours and gradually returned to initial hyperglycemic BGLs 24 hours posttreatment (FIG. 3D). Third, the ability of complexes with a two-fold polymer (LL2-insulin and L2-insulin) to regulate BGLs was enhanced, especially for that was prepared from PLL_{0.4}-FPBA_{0.6}. The BGLs of diabetic mice that received treatment with LL2-insulin were maintained below 200 mg/dL for 28 hours posttreatment and remained below the initial hyperglycemic levels even 72 hours posttreatment (FIG. 3D). Similarly, diabetic mice treated with L2-insulin also showed normoglycemic BGLs for more than 28 hours (FIG. 3D), which was significantly longer than that in diabetic mice treated with L1-insulin. While diabetic mice treated with LL1-insulin, LL2-insulin, and L2-insulin all showed lower BGLs than the original BGLs even after 30 hours, but only LL2-insulin and L2-insulin remained effective after 50 hours, though the BGLs were higher than 200 mg/dL. The blood glucose regulation capabilities of these complexes were consistent with the in vitro study, in which L1-insulin had the highest while LL2- and L2-insulin had a lowest balanced free insulin levels in 100 mg/dL glucose solution. A high balanced free insulin level could lead to fast insulin release and shorten the normoglycemia period.

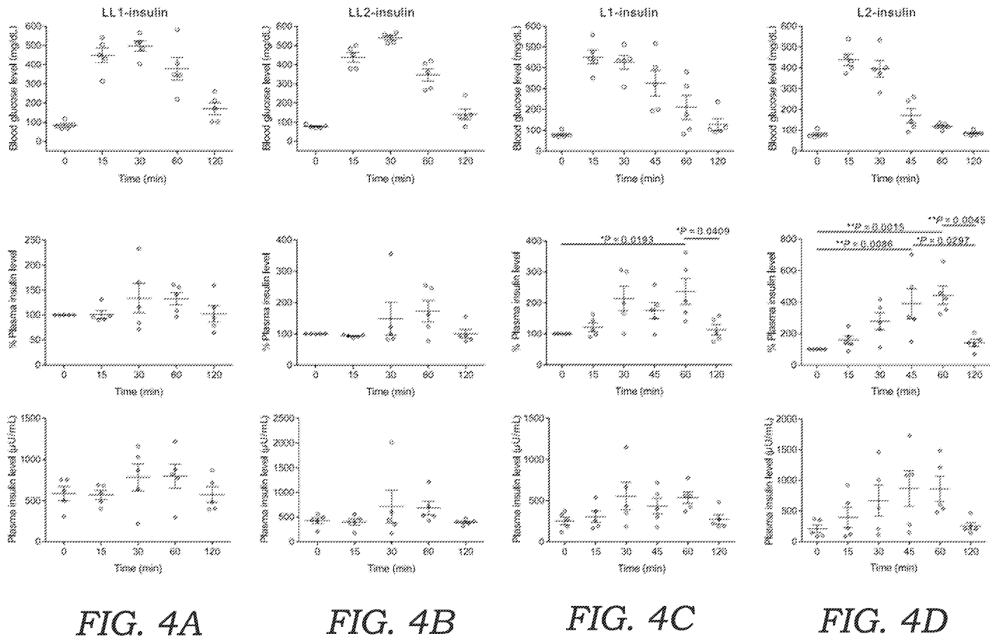

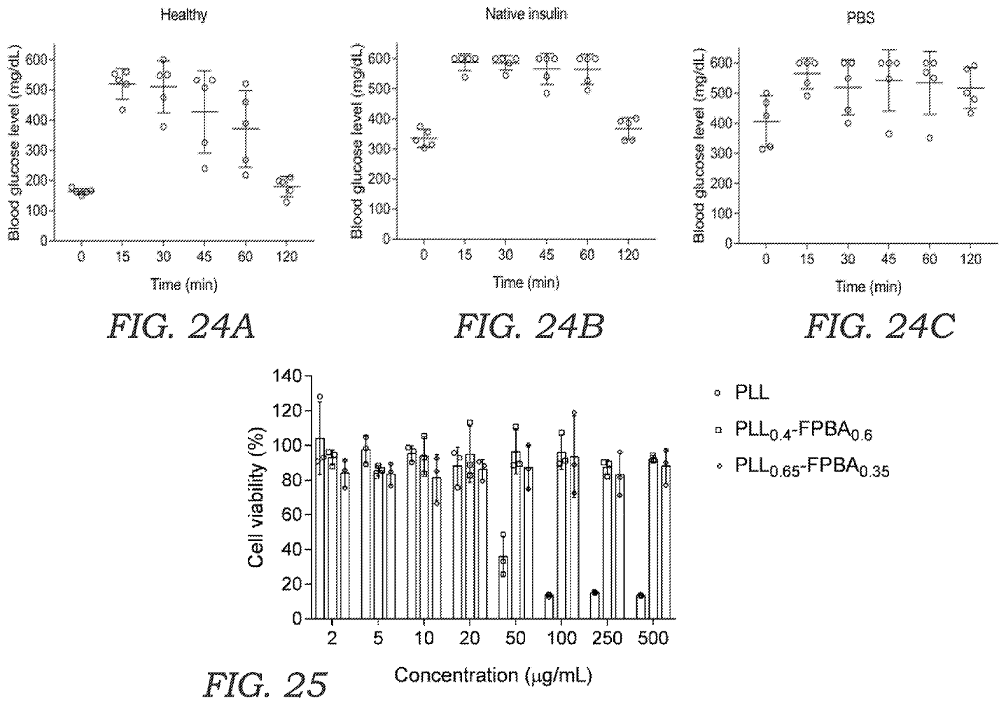

Intraperitoneal glucose tolerance tests (IPGTT) were further performed with the four insulin complexes: LL1-insulin, L1-insulin, LL2-insulin, and L2-insulin. Diabetic mice were randomly assigned to each group (n=5). Diabetic mice treated with PBS or healthy mice were used as controls. Glucose (3 g/kg) was Intraperitoneally administrated at 8 hours posttreatment. Upon administration of glucose, BGLs increased rapidly among all mice and returned to the normal range only in the healthy and complexed-treated groups (FIGS. 4A-4D and FIGS. 24A-24C). However, the associated change in plasma insulin levels varied across the mice receiving different insulin complexes. Plasma insulin levels among mice treated with LL1-insulin increased to an average of 130% compared to an average of 180% for mice treated with LL2-insulin (FIG. 4A-4B). Compared to the LL1 and LL2 insulin, the insulin complexes prepared from PLL_{0.4}-FPBA_{0.6} (L1 and L2 insulin) showed elevated glucose-responsive insulin release. After glucose administration, the plasma insulin level increased to 230% and 440% at 60 min for L1-insulin and L2-insulin treated diabetic mice, respectively (FIG. 4C-4D). In addition, the plasma insulin levels also decreased to baseline levels along with the normalization of BGLs at 120 min. Furthermore, the BGLs of mice treated with L2-insulin returned to the normal range faster than that of L1-insulin treated ones. Of note, the blood insulin levels of diabetic mice treated with L1-insulin at 8 h posttreatment were lower than that of the diabetic mice treated with LL1-insulin and LL2-insulin, which may be associated with the fast insulin release within the eight-hour period after L1-insulin injection. This is also consistent with the short euglycemia period in diabetic mice treated with L1-insulin.

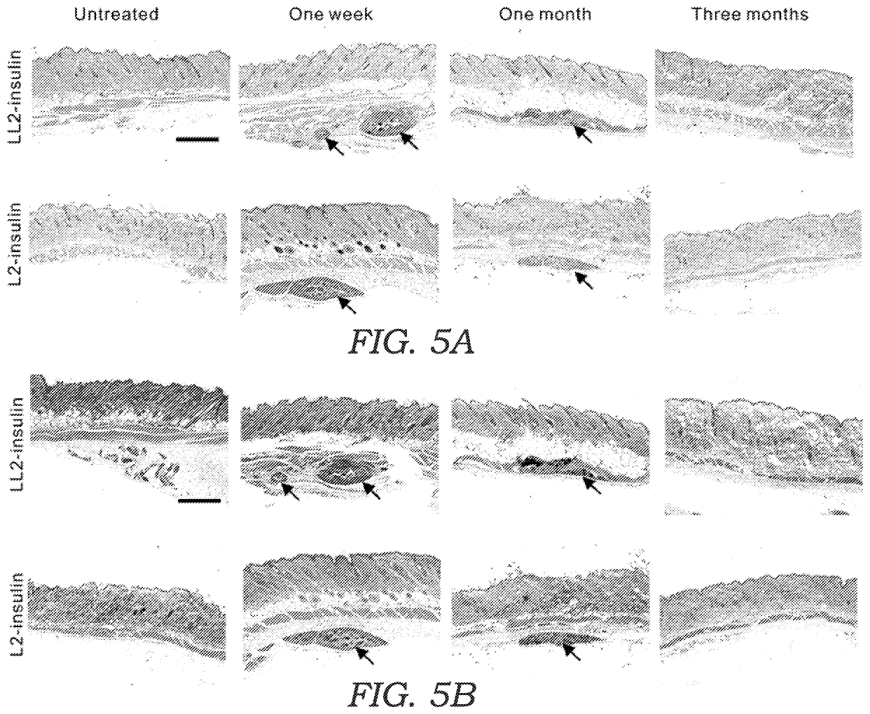

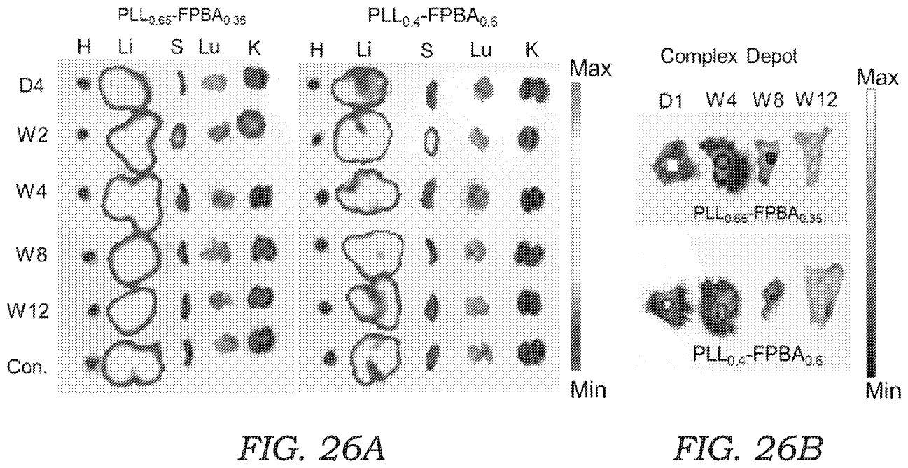

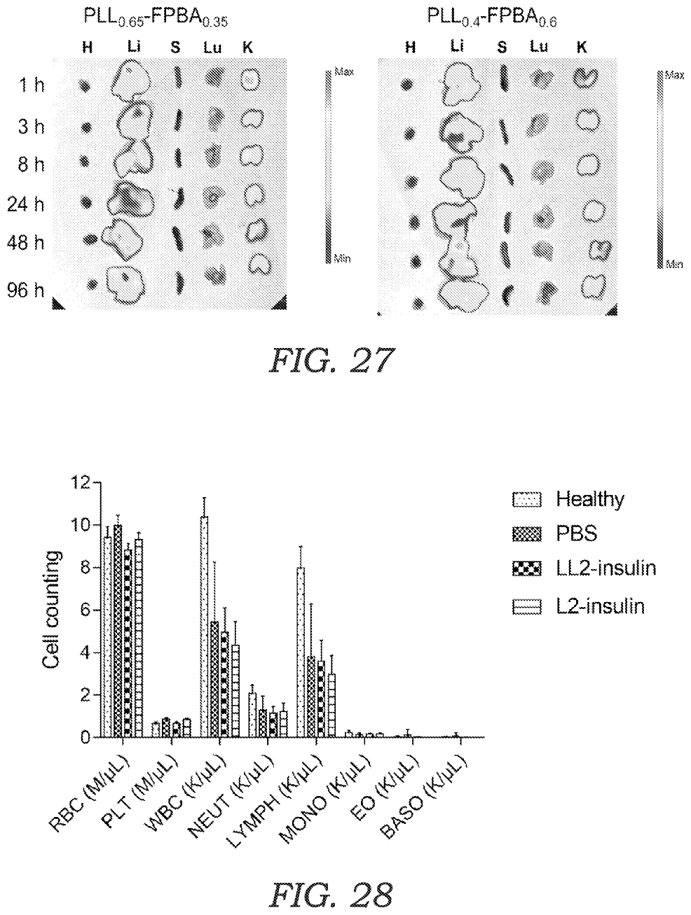

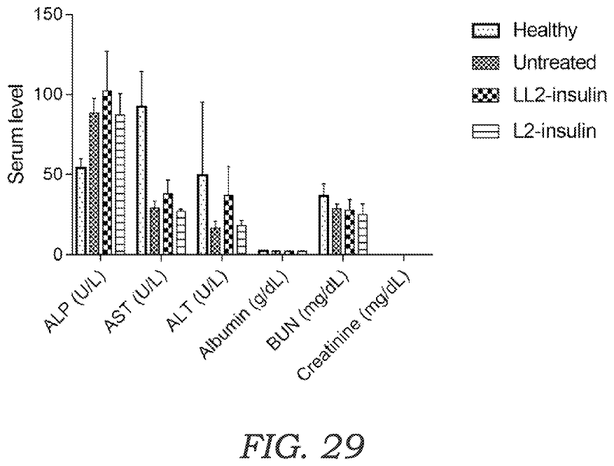

Then, in vitro cytotoxicity of PLL before and after modification by FPBA was evaluated on L929 cells. PLL_{0.65}-FPBA_{0.35} and PLL_{0.4}-FPBA_{0.6} showed negligible cytotoxicity in the studied concentration range (2 to 500 μg/mL), while unmodified PLL exhibited cytotoxicity at concentrations higher than 50 μg/mL (FIG. 25). The in vivo biocompatibility of FPBA-modified PLL was also evaluated. LL2-insulin and L2-insulin prepared with Cy5-labeled polymer were subcutaneously injected, and the biodistribution of polymers was monitored using IVIS spectrum. Both PLL_{0.65}-FPBA_{0.35} and PLL_{0.4}-FPBA_{0.6} were gradually eliminated through the liver from subcutaneous depots within three months after injection (FIGS. 26A, 26B, 27). Hematoxylin and eosin (H&E) staining results indicated that neutrophil infiltration was localized to the site of the injected complexes (FIG. 5A). In addition, the formation of collagen fibers at the injection site was minimal as observed via Masson's trichrome staining (FIG. 5B). All insulin complexes were found to be degraded or cleared entirely by three months posttreatment (FIGS. 26A, 26B), and no residual collagen fiber deposition was observed (FIG. 5B). Furthermore, no toxicity has been identified regarding the change of blood cell counts and serum biochemistry indices (FIGS. 28 and 29).

In summary, various complexes were prepared from human recombinant insulin and FPBA-modified PLL with loading efficiency higher than 90%. The complexes were prepared by leveraging the electrostatic attraction between the cationic polymers and insulin as well as the increase of entropy during the formation of polyion complexes. A higher polymer (PLL) molecular weight, a larger polymer-to-insulin ratio, and a lower FPBA-modification degree all led to reduced free insulin levels at a normoglycemia-relevant glucose solution. Glucose-stimulated insulin release from complexes was validated and dependent on the polymer MW, FPBA-modification degree, and polymer-to-insulin ratio. Among the complexes studied herein, L2-insulin exhibited the best glucose-responsiveness regarding the ratio of balanced insulin level in 400 mg/dL glucose solution to that in 100 mg/dL glucose solution. In vivo studies in type 1 diabetic mice validated that LL1-insulin, LL2-insulin, L1-insulin, and L2-insulin all had the ability to prolong anti-hyperglycemic effect of native insulin, especially for LL2-insulin and L2-insulin, both of which achieved extended normoglycemia for more than 20 hours and remained effective even at 72 hours posttreatment. This prolonged treatment efficacy is consistent with their ultra-low free insulin level in glucose solution at 100 mg/dL. Furthermore, in vivo IPGTT-stimulated insulin release performance of subcutaneous L2-insulin depot was found to be the best among the four complexes, which is in agreement with its highest ratio of balanced free insulin in 400 mg/dL glucose solution to that in 100 mg/dL glucose solution among the complexes in this study. From a biocompatibility perspective, all complexes were shown to be absent from subcutaneous tissue samples after three months, and no obvious biocompatibility issues were identified. Overall, these results clarify the relevance between in vitro and in vivo glucose-responsive performance. It was found that the differences in insulin release rates and equilibrated free insulin levels across normoglycemic and hyperglycemic conditions were critical for maximizing the in vivo glucose-responsive performance of this type of insulin delivery systems. As such, these results provide important data for the continued optimization of future glucose-responsive insulin delivery systems.

Materials and Methods

Poly-L-lysine hydrobromide with various MW was purchased from Sigma-Aldrich. Dialysis tube membrane (MWCO=3500 Da) was purchased from Spectrum Laboratories. N-hydroxysuccinimide (NHS) and 4-carboxy-3-fluorobenzeneboronic acid (FPBA) were purchased from Fisher Scientific. Recombinant human insulin was purchased from ThermoFisher Scientific (Catalog No. A113811IJ). Other reagents were purchased from Sigma-Aldrich. NHS ester of FPBA (FPBA-NHS) was prepared as previously described in Wang et al.

Synthesis of FPBA-modified PLL, with PLL_{0.4}-FPBA_{0.6} (30-70K) as an example.

PLL (100 mg) was dissolved in PBS (0.01 M, pH=7.4, 10 mL), to which FPBA-NHS (120 mg) dissolved in DMSO (5 mL) was added dropwise while the pH was kept around 7. After the addition of FPBA-NHS solution, the reaction was stirred for another 30 min before dialysis in deionized water (4 L). The obtained mixture was lyophilized, and a white solid was obtained. The product was characterized by ¹H-NMR to determine the degree of FPBA modification.

Preparation of insulin labeled with rhodamine B (RhB-insulin). Rhodamine B isothiocyanate (5 mg) was dissolved in DMSO (1 mL) and then added to the insulin solution (0.1 M Na₂CO₃, 50 mg/mL, 2 mL). The mixture was stirred at room temperature for two hours before dialysis in deionized water (3×4 L). After lyophilization, purple RhB-insulin was obtained. Cyanine 5 (Cy5) labeled PLL-FPBA or native insulin was prepared similarly.

Preparation of insulin complex, with PLL_{0.4}-FPBA_{0.6} as an example. Both native insulin (10 mg/mL) and PLL_{0.4}-FPBA_{0.6} (10 mg/mL) were prepared beforehand. Then, both solutions (100 μL) were mixed and one drop of NaOH (1N) was added to bring the pH to 7.4. Subsequently, PBS (pH=7.4, 1 mL) was added, and the mixture was centrifuged to remove unloaded insulin. The final insulin complex was dispersed in PBS (10 mM, pH=7.4) at 1 mg/mL (insulin equivalent). The complex was used immediately for subsequent experiments. Other complexes with varied polymers or polymer-to-insulin ratios were prepared in a similar procedure. The insulin level in the supernatant was measured using Coomassie protein assay reagent and calculated using a standard curve. The insulin loading efficacy was calculated accordingly.

Characterization of complex particles. Hydrodynamic size and zeta-potential of complexes were measured on a ZETAPALS (Brookhaven Instruments Corporation). The complexes were suspended in PBS with a final insulin concentration of 0.5 mg/mL. The zeta-potential of the complex at various glucose solutions was measured after adding glucose (0.4 g/mL) to complex suspension and incubating for 5 min. of note, the particles were polydispersed and easy to precipitate, especially after the addition of glucose. Before observing the complex by SEM (ZEISS Supera 40VP) and TEM (T12 Quick CryoEM and CryoET (FEI)), the LL2-insulin complex was centrifuged, and PBS was replaced by deionized water. The concentration of complex was equivalent to 0.5 mg/mL insulin. The TEM sample was stained by phosphotungstic acid (2%).

3-(4,5-dimethylthiazol-2-yl)-2,5-diphenyltetrazolium bromide (MTT) assay. The L929 murine fibroblast cell line was purchased from ATCC. RPMI 1640 medium was supplemented with heat-inactivated fetal bovine serum (10%), penicillin (100 units/mL), and streptomycin (0.1 mg/mL) and used to grow the cells. For cytotoxicity assay, cells were seeded into a 96-well plate (100 μL medium, 10, 000 cells per well) for 24 hours before the addition of polymer solution or suspension in culture medium (100 μL) with series concentrations. The cells were incubated with polymers for another 24 hours. Then, the culture medium was replaced with fresh medium with 0.75 mg/mL MTT (100 μL) for another three hours. After the removal of the MTT medium, DMSO (200 μL) was added. After gently shaking for 10 min, the absorbance of each well was measured at 562 nm using a microplate spectrophotometer. Each polymer concentration was tested in triplicate.

In vitro glucose-binding ability study. Complexes (L1-insulin, LL1-insulin) were suspended in PBS 7.4 (1 mL) with the final suspension containing 1 mg/mL PLL-FPBA. Then, glucose (0.4 g/mL) was added to each vial to obtain initial glucose concentrations of 100, 200, and 400 mg/dL. At predetermined time point, the suspension was obtained, and the glucose concentration was measured using a glucose meter (Clarity, BG1000) with the high limit of 600 mg/dL. A standard curve was established for calibration. The glucose solution with concentration over 200 mg/dL was diluted in an equal volume of PBS before measurement.

In vitro insulin release study. The complex suspension was prepared by adding PBS to complex. Complex prepared from native insulin and complex was suspended in PBS (pH=7.4, 1 mg/mL), and allocated to Eppendorf tubes. Into these tubes, glucose (0.4 g/mL) was added to obtain varied glucose concentrations (0, 100, 200 and 400 mg/dL). These tubes were incubated at 37° C. At timed intervals, the complex suspension was withdrawn and centrifuged. The clear supernatant was used to measure the insulin concentration using Coomassie protein assay reagent via first establishing a standard curve. Of note, the supernatant of the complex suspension was measured before the addition of glucose and had absorbance almost comparable to blank PBS and was set as zero point. Moreover, both PLL_{0.4}-FPBA_{0.6} and PLL_{0.65}-FPBA_{0.35} are insoluble in PBS at pH 7.4 with glucose concentrations in the range of 0 to 400 mg/dL, indicating minimal interference from polymers.

In vivo blood glucose-regulation study in type 1 diabetic mice. All animal procedures were performed following the Guidelines for Care and Use of Laboratory Animals of University of California, Los Angeles. Streptozotocin-induced diabetic mice were purchased from Jackson Laboratory. Mice were fed with standard diet and exposed to a 12-hour light and 12-hour dark environment. Mice with BGLs higher than 300 mg/dL were selected for the study. Diabetic mice (n=5 to 10) were allocated to groups treated with native insulin and various complexes. The insulin equivalent dose of each complex was determined to be 1.5 mg/kg (43 U/kg). The blood glucose was monitored before and after treatment until the blood glucose returned to initial levels. The blood samples were taken from the tail tips and plasma glucose concentration was measured by a glucose meter (Aviva, ACCU-CHEK).

Intraperitoneal glucose injection-induced insulin release study. Diabetic mice (n=5) were randomly assigned to be treated with various insulin complexes (1.5 mg/kg). 8 hours posttreatment, these mice were intraperitoneally injected with glucose (3 g/kg). Blood samples (40 μL) were extracted and transferred into Eppendorf tubes pretreated with EDTA. The blood was collected just before glucose injection and at predetermined timed intervals after the glucose injection. The obtained blood was centrifuged, and the plasma insulin level was quantified using a human insulin enzyme-linked immunosorbent assay (ELISA) test (Invitrogen).

Statistical analysis. One-way ANOVA with Tukey post-hoc tests and Two-way ANOVA were used to carry out multiple comparisons.

While embodiments of the present invention have been shown and described, various modifications may be made without departing from the scope of the present invention. The invention, therefore, should not be limited, except to the following claims, and their equivalents.

1. A therapeutic glucose-responsive material comprising a poly-L-lysine (PLL) polymer modified with 4-carboxy-3-fluorophenylboronic acid (FPBA) (PLL-FPBA) that is loaded with insulin to form a polymer-insulin complex.

2. The therapeutic glucose-responsive material of claim 1, wherein the modified polymer PLL-FPBA is loaded with about an equal (weight basis) amount of insulin.

3. The therapeutic glucose-responsive material of claim 1, wherein the material has about twice (weight basis) the amount of modified polymer PLL-FPBA as the amount of insulin.

4. The therapeutic glucose-responsive material of claim 1, wherein the material comprises between about 1 to about 2 times (weight basis) modified polymer PLL-FPBA as the amount of insulin.

5. The therapeutic glucose-responsive material of claim 1, wherein the modified polymer has the formula PLLₓ-FPBA_{y}, wherein x is in the range of about 0.2 to about 0.9 and y is in the range of about 0.8 to about 0.1.

6. The therapeutic glucose-responsive material of claim 1, wherein the modified polymer has the formula PLLₓ-FPBA_{y}, wherein x is in the range of about 0.4 to about 0.65 and y is in the range of about 0.6 to about 0.35.

7. The therapeutic glucose-responsive material of claim 1, wherein the material is maintained at a pH of about 7.4.

8. The therapeutic glucose-responsive material of claim 1, wherein the PLL has a molecular weight within the range of 30-70 kg/mol.

9. A kit comprising:
an injection device; and
a therapeutic glucose-responsive material comprising a poly-L-lysine (PLL) polymer modified with 4-carboxy-3-fluorophenylboronic acid (FPBA) forming a complex with insulin.

10. A method of using the therapeutic glucose-responsive material of claim 1 comprising:
delivering a volume of the therapeutic glucose-responsive to a subject.

11. The method of claim 10, wherein the material is delivered by injection.

12. (canceled)

13. The method of any claim 10, wherein the subject is a type 1 diabetic.

14. The method of any claim 10, wherein the subject is a type 2 diabetic.

15. A method of altering glucose levels within a subject comprising:
delivering a volume of therapeutic glucose-responsive material subcutaneously or intramuscularly to a subject, the glucose-responsive material comprising a poly-L-lysine (PLL) polymer modified with 4-carboxy-3-fluorophenylboronic acid (FPBA) (PLL-FPBA) in a complex with insulin.

16. The method of claim 15, wherein normoglycemia is maintained in the subject for at least 10 hours post-delivery of the therapeutic glucose-responsive material.

17. The method of claim 15, wherein normoglycemia is maintained in the subject for at least 28 hours post-delivery of the therapeutic glucose-responsive material.

18. A method of making a therapeutic glucose-responsive material comprising;
modifying poly-L-lysine (PLL) polymer with 4-carboxy-3-fluorophenylboronic acid (FPBA) to form a modified polymer (PLL-FPBA);
mixing the PLL-FPBA with insulin in an acidic solution followed by rapidly adjusting the pH of the mixture to around 7.4 to load insulin in the PLL-FPBA.

19. The method of claim 18, wherein the modified polymer PLL-FPBA is loaded with about an equal (weight basis) amount of insulin.

20. The method of claim 18, wherein the amount (weight basis) of modified polymer PLL-FPBA is about twice the amount of insulin.

21. The method of claim 18, wherein the material comprises between about 1 to about 2 times (weight basis) modified polymer PLL-FPBA as the amount insulin.

22. The material of claim 18, wherein the modified polymer has the formula PLLₓ-FPBA_{y}, wherein x is in the range of about 0.2 to about 0.9 and y is in the range of about 0.8 to about 0.1.

23. The method of claim 18, wherein the modified polymer has the formula PLLₓ-FPBA_{y}, wherein x is in the range of about 0.4 to about 0.65 and y is in the range of about 0.6 to about 0.35.

24. The method of claim 18, wherein the PLL has a molecular weight within the range of 30-70 kg/mol.
